(19) United States
(12) Patent Application Publication
(10) Pub. No.: US 2020/0410889 A1
(43) Pub. Date: Dec. 31, 2020

(54) CONCUSSION AVOIDANCE TRAINING SYSTEM AND METHOD

(71) Applicant: Chiming Huang, Shawnee Mission, KS (US)

(72) Inventor: Chiming Huang, Shawnee Mission, KS (US)

(21) Appl. No.: 16/915,250

(22) Filed: Jun. 29, 2020

Related U.S. Application Data
(60) Provisional application No. 62/868,046, filed on Jun. 28, 2019.

(51) Int. Cl. G09B 19/00; G09B 9/00; A61N 1/38; A61N 1/36

(52) U.S. Cl. CPC G09B 19/00; G09B 9/00; G06F 3/167; A61N 1/36028; A61N 1/38

(57) ABSTRACT

A system and method for reducing and preventing head injuries using a combination of hardware and software. Elevated neck stiffness can be learned through the system, device, and the method described in the present invention by delivering and pairing a certain appropriate sensory cue (SnC) with a certain appropriate significance cue (SgC). the system, device, and method are used to generally facilitate and validate an increase in neck stiffness to prevent or reduce the likelihood of concussion upon impact. The system employs a combination of hardware and software, such as using modified virtual reality (VR) headsets (in both hardware and software), to deliver visual, auditory, and other cues (somatosensory, vestibular, etc.) as SnC. The system also employs a combination of hardware and software, such as using modified virtual reality (VR) headsets (in both hardware and software), to deliver somatosensory, vestibular, visual, auditory, and other cues as SgC.

### CROSS-REFERENCE TO RELATED APPLICATION

This application claims priority in U.S. Provisional Patent Application No. 62/868,046, filed Jun. 28, 2020, which is incorporated herein by reference.

### BACKGROUND OF THE INVENTION

### 1. Field of the Invention

The present invention relates generally to a concussion solution and method for use thereof, and more specifically to a concussion avoidance training (CAT) system and method.

### 2. Description of the Related Art

Closed-head traumatic brain injury (TBI) is typically a result of the brain impacting the interior of the skull. Forces acting on the body or the head generally accelerate the brain. High positive acceleration or negative acceleration may cause the brain to contact the insides of the skull with enough force to cause brain injury. In addition, these accelerations set up transient pressure and strain gradients within the soft neuronal tissue of the brain. These gradients can sometimes bring about dramatic disruptions in neuronal metabolism and function at the cellular level without obvious or noticeable macroscopic movement of the brain. Such disruptions may take the form of diffuse axonal injuries or DAI which is manifested at the cellular or sub-cellular level. Disruptions may also take the form of glutamatergic neurotoxicity or excite-toxicity which is manifested at the neurotransmitter level. The types of brain injury may also be categorized as blast TBI, concussive TBI, or mild TBI, etc. Blast TBI may be experienced by military or law enforcement personnel while on patrol or traveling in a vehicle. Concussions which are also synonymous with mild TBI or mTBI may be suffered by athletes in sports such as hockey, boxing, soccer, or American football. Mild TBI may be experienced by anyone suffering a fall, a vehicular accident, a bicycle accident, or the like.

The concussion threshold for an individual may change over time. The period of time may be long and span over many years during which development or maturation occurs in children and adolescents. Or it may be short, spanning over a matter of minutes or days as it is now known that a person is likely to be more vulnerable to a second concussion immediately after a concussion. Or it may be shorter still, spanning over a matter of seconds or fractions of a second as concussions are inopportune events when the impact force catches the head-and-neck in the moment when the neck stiffness is low. The matter of time over seconds or less is therefore relevant as the consequences of a head impact event can be disastrous especially when the impact occurs while the person does not know, is not prepared, or is otherwise unaware of the impending impact. Concussive thresholds are therefore complex, individualized or personalized, dependent on the magnitude and directionality of the impact force with respect to the framework of pitch-roll-yaw axes as the three rotational degrees of freedom of the head-and-neck, and also dependent upon the neck stiffness at the moment of impact.

Systems that have been developed for preventing concussions for players of American football may include improved material and construction of football helmets, one or more acceleration sensors or head-impact-measurement devices coupled to a football helmet and one or more mechanisms connecting the helmet to shoulder pads. The systems may further include a processing element that locks the coupling or connecting mechanisms when the acceleration measured by the sensors exceeds a certain constant value believed to be a threshold beyond which a concussion to the player may occur.

Heretofore there has not been available a system or method for concussion avoidance training (CAT) with the advantages and features of the present invention.

The head acceleration [a] due to an impact force [f] is determined by Newton's law f=ma, where the effective head mass [m] is dependent on the neck stiffness at the moment of impact. A stiffer neck can lead to smaller head accelerations during impact by increasing the effective head mass [Viano D C, Casson I R, Pellman E J (2007) Concussion in professional football: biomechanics of the struck player—part 14, Neurosurgery 61:313-327. doi:10.1227/01.NEU.0000279969.02685.DO]. Humans most certainly have control over their head-and-neck muscles, therein lies the opportunity for technologies and training strategies to reduce concussion risk.

This invention is a system and device that allow a person to reflexively develop a stiffer neck at the moment of impact, thereby increasing the effective mass of the head, decreasing impact-induced head angular accelerations, and reducing concussive risk.

In essence, the system and device works to allow the brain of a person to build de novo a neuronal pathway which then causes the person to automatically and reflexively to act in ways to protect themselves. Here, the system and device allows the brain of a person to build de novo a neuronal pathway which then causes the person to automatically and reflexively elevate neck stiffness prior to or at the moment of impact, thereby reducing mTBI risk.

### BRIEF SUMMARY OF THE INVENTION

The present invention generally provides a system and method for reducing and preventing head injuries using a combination of hardware and software. Elevated neck stiffness can be learned through the system, device, and the method described in the present invention by delivering and pairing a certain appropriate sensory cue (SnC) with a certain appropriate significance cue (SgC). The present invention comprises a system, device, and method to generally facilitate and validate an increase in neck stiffness to prevent or reduce the likelihood of concussion upon impact.

The present invention employs a combination of hardware and software, such as using modified virtual reality (VR) headsets (in both hardware and software), to deliver visual, auditory, and other cues (somatosensory, vestibular, etc.) as SnC.

The present invention employs a combination of hardware and software, such as using modified virtual reality (VR) headsets (in both hardware and software), to deliver somatosensory, vestibular, visual, auditory, and other cues as SgC.

The present invention employs a combination of hardware and software, such as using modified virtual reality (VR) headsets (in both hardware and software), to measure and monitor the muscular activities of head-and-neck muscles and neck stiffness as reflexive activities or R.

The present invention employs a combination of hardware and software, such as using modified virtual reality (VR) headsets (in both hardware and software), to measure and monitor the muscular activities of head-and-neck muscles and neck stiffness as learned behaviors or LB.

Related technology such as that disclosed in U.S. Pat. Nos. 8,961,440 and 9,226,707 for a “Device and System to Reduce Traumatic Brain Injury,” are owned by the same inventor as the present application and are incorporated herein by reference.

### BRIEF DESCRIPTION OF THE DRAWINGS

The drawings constitute a part of this specification and include exemplary embodiments of the present invention illustrating various objects and features thereof.

FIG. 1 is a block diagram showing components for practicing an embodiment of the present invention.

FIG. 2 is a block diagram showing components which may be implemented to practice an embodiment of the present invention.

FIG. 3 is a diagram identifying head and neck muscle elements which may be affected by practicing an embodiment of the present invention.

FIG. 4 is a block diagram showing the relationship between components used in practicing an embodiment of the present invention.

FIG. 5 is a side elevational view of components used in practicing an embodiment of the present invention in a typical environment of a user's head and neck.

FIG. 6 is a rear elevational view thereof, showing sensor components placed about the user's head and neck.

FIG. 7 is a rear three-dimensional isometric view thereof.

FIG. 8 is a flowchart diagramming steps taken in practicing an embodiment of the present invention.

### DETAILED DESCRIPTION OF THE PREFERRED EMBODIMENTS

### I. Introduction and Environment

As required, detailed aspects of the present invention are disclosed herein. However, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

Concussions or mild traumatic brain injuries (mTBI) are consequences of head angular accelerations above certain thresholds due to impact forces acting on one's head or body. The CDC (Centers for Disease Control and Prevention) estimates that 1% of the US population suffers from concussions every year [CDC (2018) Rates of TBI-related Emergency Department Visits, Hospitalizations, and Deaths—United States, 2001-2010, (CDC tracks data on traumatic brain injuries in US on their website https://www.cdc.gov/traumaticbraininjury/data/rates.html)]. That chance jumps to ˜10% per season for a high school boy playing football or a girl playing soccer [Broglio S P, Schnebel B, Sosnoff J J, Shin S, Fend X, He X, Zimmerman J (2010) Biomechanical properties of concussions in high school football, Med Sci Sports Exerc. 42 (11): 2064-2071. doi: 10.1249/MSS.0b013e3181dd9156; Comstock R D, Currie D W, Pierpoint L A (2015) SUMMARYREPORT. NATIONAL HIGH SCHOOL SPORTS-RELATED INJURY SURVEILLANCE STUDY. 2014-2015 School Year (www.ucdenver.edu/academics/colleges/ . . . /Original%20Report_%202014_15.pdf);, Delaney J S, Lacroix V J, Leclerc S, Johnston K M (2000). Concussions during the 1997 Canadian Football League season. Clin J Sport Med 10:9-14 (https://www.ncbi.nlm.nih.gov/pubmed/10695844); Kaut K P, DePompei R, Kerr J Congeni J (2003). Reports of head injury and symptom knowledge among college athletes: implications for assessment and educational intervention, Clin J Sport Med 13:213-221. PMID: 12855923; McCrea M, Hammeke T, Olsen G, Leo P, Guskiewicz K (2004) Unreported concussion in high school football players: implications for prevention. Clin J Sport Med 14:13-17. PMID: 14712161; Meehan W P, Bachur R G (2009) Sport-related concussion. Pediatrics 123:114-123. doi: 10.1542/peds.2008-0309; Rowson S, Duma S (2013) Brain injury prediction: Assessing the combined probability of concussion using linear and rotational head acceleration, Ann Biomed. Engineering, 41:873-882. doi: 10.1007/s10439-012-0731-0]. Concussions can lead to chronic traumatic encephalopathy (CTE), which has no known cure, can occur as early as the second or third decade of life, and is linked to excessive aggression, accelerated dementia, depression, and suicide [Gavett B E, Stern R A, McKee A C (2011) Chronic traumatic encephalopathy: a potential late effect of sport-related concussive and sub-concussive head trauma. Clin Sports Med 30:179-188. doi: [10.1016/j.csm.2010.09.007], McKee A C, Cantu R C, Nowinski C J Hedley-Whyte E T, Gavett B E, Budson A E, Santini V E, Lee H S, Kubilus C A, Stern R A (2009) Chronic traumatic encephalopathy in athletes: progressive tauopathy after repetitive head injury. J Neuropathol Exp Neurol 68:709-735. doi:10.1097/NEN.0b013e3181a9d503].

There are 4.8 million football players in elementary, middle, and high schools in the US [Guskiewicz K M, Weaver N L, Padua D A, Garrett W E (2000) Epidemiology of concussion in collegiate and high school football players, Am J Sports Med 28: 643-650]. Similar demographics exist in soccer. As stated earlier, the rate of concussion for a high school football player is about 10% per season, roughly ten times that of the general U.S. population. The concussion rate per 10,000 athletic exposures in high school girls' soccer is 8.19, which is right up there with 10.40 in high school football [Dompier T P, Kerr Z Y, Marshall S W, Hainline B, Snook E M, Hayden R, Simon J E (2015) Incidence of concussion during practice and games in youth, high school, and collegiate American football players, JAMA Pediatr. 169: 659-665].

A person's concussive threshold changes from moment to moment depending on his or her neck stiffness. The head acceleration [a] due to an impact force [f] is determined by Newton's law f=ma, where the effective head mass [m] is dependent on the neck stiffness at the moment of impact. Neck stiffness is a measure of how the neck resists a force while neck strength is how the neck exerts force. Although neck strength is a significant predictor of concussions [Collins C L, Fletcher E N, Fields S K, Kluchurosky L, Rohrkemper M K, Comstock R D, Cantu R C (2014) Neck strength: A protective factor reducing risk for concussion in high school sports, J Primary Prevent 35:309-319. doi: 10.1007/s10935-014-0355-2] neck strength training had limited effects on head-and-neck kinematics in head-impact events in soccer players [Mansell J, Tierney R T, Sitler M R, Swanik K A, Stearme D (2005) Resistance Training and Head-Neck Segment Dynamic Stabilization in Male and Female Collegiate Soccer Players,

J Athl Train 40(4): 310-319. PMID: 16404453]. Football players with great neck strength are concussed with regularity. Addressing neck strength alone may not be sufficient in concussion risk assessments [Mihalik J P, Guskiewicz, K M, Marshall S W, Greenwald R M, Blackburn T, Cantu R C (2011) Does cervical muscle strength in youth ice hockey players affect head impact biomechanics? Clin J Sport Med 21: 416-420. doi: 10.1097/JSM.0B013E31822C8A5C]. The significance of neck stiffness over neck strength in mTBI can become evident in head butts in animals with strong territorial instincts. It is particularly instructive when such encounters occur in animals with large differences in neck girth, head, and body size. An impact force to the head will have to move part of the body as well as the head if the neck is stiff. As some of the impact energy is being dissipated to move the body, less of the impact energy will go to the head. A stiffer neck therefore can lead to smaller head accelerations during impact by increasing the effective head mass [Viano et al 2007]. The lesson is that a football player may have a better chance of walking away from a collision without mTBI if he learns from the goat. Humans most certainly have control over their head-and-neck muscles, therein lies the opportunity for technologies and training strategies to reduce concussion risk.

A method for the learned acquisition of a stiff neck for concussion avoidance training (CAT) has been derived in the present application. Similar learned behaviors in the form of reflexive muscular contractions routinely occurs in many practical examples generally referred to as sports training. Examples of sports training include learning to ride a bicycle when the rider shifts the center of gravity of his body quickly in order to balance oneself on two wheels. The subtle, learned, quick movements and muscular contractions associated with the learned behavior occur automatically at a subconscious level and with precise timing.

Sports training learning typically involves a limited number of muscles within a localized region of the body and their control (although in some cases can also involve a large number of muscles distributed over a large area of the body such as playing piano, or playing tennis). One unique sports-training aspect of the present invention is that the relevant muscles in the CAT described in the present invention are largely, if not all, in the head-and-neck region. A second unique aspect of the CAT is that it calls for simultaneous, co-contractions of all agonist as well as antagonist muscles—not typically done in sports training.

During normal movements, contractions of agonist muscles and antagonist muscles on the two sides of a joint must be 180 degree out-of-phase or else no movement can be expected. Agonist and antagonist muscles, therefore, are programmed by the brain and spinal cord nerve cells to contract and relax out-of-phase. Co-contraction, therefore, requires training as well as de novo neuronal circuits. It is to be noted, however, no new neurons are needed. The de novo neuronal circuits involves many new synapses.

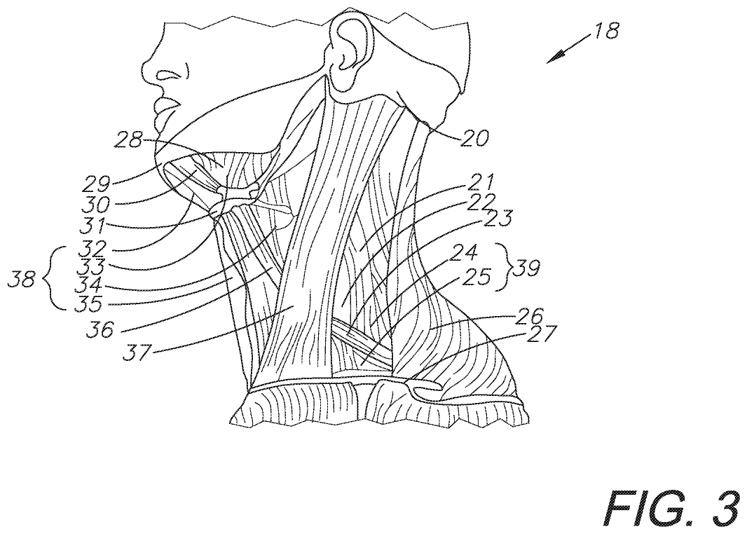

The head-and-neck muscles are the hardware components of our CAT. Knowledge on head-and-neck musculature is therefore important to designing our CAT. In the head-and-neck region, there are approximately two dozen muscles involved in neck stiffness. About fewer than half a dozen of these muscles are responsible for most of the gross head-and-neck movement. The speed of contraction of these muscles 18 are generally of the order of 25 to 300 milliseconds as shown in FIG. 3. The muscles as identified in FIG. 3 are as indicated in the following Table A:

TABLE A
(Ref. FIG. 3)

| Reference Numeral | Muscle |
|---|---|
| 20 | Processus mastoideus |
| 21 | m. scalenus medius |
| 22 | m. scalenus anterior |
| 23 | m. omohyoideus (venter inferior) |
| 24 | Occipital triangle |
| 25 | Subclavian triangle |
| 26 | m. trapezius |
| 27 | clavicular |
| 28 | m. mylohyoideus |
| 29 | Mandibular |
| 30 | m. digastricus |
| 31 | Os hyoideum |
| 32 | Submental triangle |
| 33 | Submandibular triangle |
| 34 | Carotid triangle |
| 35 | Muscular triangle |
| 36 | m. omohyoideus (venter superior) |
| 37 | m. sternocleid omastoideus |

Reference numeral 38 generally identifies those muscles making up the anterior triangle, and reference numeral 39 generally identifies those muscles making up the posterior triangle.

If the head-and-neck muscles are the hardware components of a person's CAT, the brain and the cerebellum in particular provide the software components of the CAT. Knowledge on the cerebellum is therefore important to designing our CAT. The applicant is a neuroscientist whose R&D has focused on the cerebellum supported by the applicant's publications in peer-reviewed scientific journals from 1982 to 2020.

According to current understanding in cerebellum neurobiology, the cerebellum provides the bulk and the necessary ‘software’ support in sports training. In general, the cerebellum evaluates ongoing challenges in life, covering three main functional areas—balance, movement, and mental gymnastics. This characterization is largely based on the three anatomical subdivisions of the cerebellum—the vestibulocerebellu, the spinocerebellum, and the neocerebellum [Purves D, Augustine G J, Pitzpatrick D, Hall W C, LaMantia A, Mooney R D, POlatt ML, White L E (2017) Neuroscience, 6th ed., Oxford University Press (London)]. Regardless of the specific nature of the challenges of life, the fundamental neuronal mechanisms to evaluate life challenges and provide solutions are the same. The manner in which the cerebellum contributes to our CAT is now briefly described below.

For the cerebellum to help in our CAT, the cerebellum first identifies the challenge by recognizing a significance cue. When a child is on the seat of a bicycle for the first time, the cerebellum immediately identifies the challenge to maintain his or her balance and avoid a crash. The loss of balance and the sensation of going into a crash are all parts of the significance cue. The significant cue is processed by the cerebellar climbing fibers, the inferior olivary complex, and related cerebellar structures.

Second, the cerebellum identifies among all existing hardware such as muscles and all existing software such as brain circuits that are candidates for meeting the challenge. In learning to ride a bicycle, the child figures out a series of actions taking advantage of his or her existing hardware and software capabilities. Indeed, he or she calls upon the things learned years ago when he or she started walking on two legs. The key is of course to recognize the very initial stages of imbalance and make corrections immediately before the imbalance becomes a crash. Recognize that very initial signs of danger is to recognize a certain set of related and relevant sensory cue, an action processed and supported by the cerebellar mossy fiber system and related cerebellar structures.

Third, the cerebellum begins to modify and alter synaptic connections to improve the efficiency of the entire process, starting from the processing of significance cue, the sensory cue, and the mobilization of new synaptic circuits to come up with solutions to meet the challenge. These actions now involve modifications in neurotransmitter secretions, the type and sensitivities of neurotransmitter receptors, the elimination of synapses, the construction of new synapses as well additional downstream actions that also involves DNA expressions, extending to brain parts beyond the cerebellum.

We now summarize our description on the CAT up to this point. Humans face new challenges on a regular basis. The cerebellum has evolved to handle such challenges by identify significance cues and sensory cues. In time, actions are taken and the person rises up to the challenge. The entire set of events are often taking place at a subconscious level. The person goes through sports training and seems to become better at a sport effortlessly. The present application concerns a system that facilitate the identification of significant cue and the sensory cue. At a level, we argue that the CAT itself is indeed like a set of training wheels that facilitate the type of sports training which is needed for reducing concussion risk.

An added point on our CAT is that, like riding a bicycle, once you learned it, it will stay with you simply because you now are endowed with a set of neuronal circuitries that will provide such a function. You will not forget it just as most people will not easily forget how to ride a bicycle.

The degree of difficulty or the ease of a certain sports training is dependent upon the number of muscles involved as well as the required precision of the activation and de-activation of the muscles involved. For example, learning to play piano or learning to hit a tennis ball proficiently will take more effort and time than learning to ride a bicycle because the learned task or learned behavior requires considerably more precision in the control of timing of movements. Variations in the timing of how one shifts his or her body weight may not immediately cause a fall. Similar variations in the timing of a forehand swing, however, may cause the forehand to be significantly off in direction and in power. The task is difficult typically because more muscles are involved in the learned behavior and the required timing of the activation and inactivation of the muscles involved is more precise.

In the present invention, and for the purpose of illustration, the description of the method is divided into three components: (a) the presentation of the appropriate significance cue (SgC) 4, (b) the presentation of the appropriate sensory cue (SnC) 6 and (c) the measurement and the monitoring of reflex (R) 14 and learned behavior (LB) 16.

Preliminary studies in the lab of the applicant and in the lab of other investigators studying sports training have suggested that training head-and-neck muscles for stiffness involves approximately one week to ten days of daily training sessions of 30-minute each [Christian K M and Thompson R F (2003) Neural substrates of eye blink conditioning: Acquisition and retention, Learning and Memory 10:427-455. doi: 10.1101/lm.59603].

It is, however, possible or probable, that the concussion avoidance training method (CAT) described in the present invention will take more, or less time depending on the age, gender, and other factors such as the sports, e.g. football, hockey, soccer, etc.

### II. Preferred Embodiment Concussion Avoidance and Training (CAT) System 2

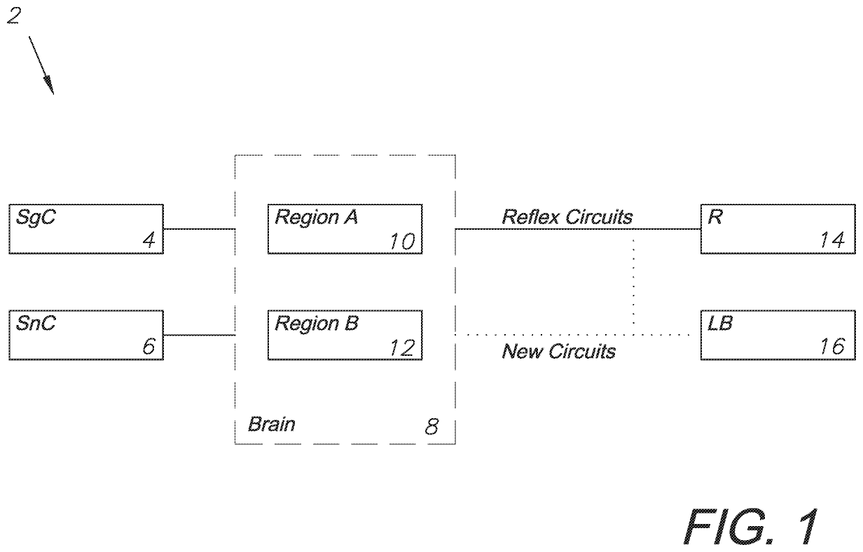

FIG. 1 diagrams the major components of the concussion avoidance and training system 2. There are three major components in the method of the present invention. They are the delivery of SgC 4, SnC 6, and the measurement and monitoring of Reflex (R) 14 and Learned Behavior (LB) 16. The purpose is to modify the reflex circuits from the brain 8, based in region A 10 and region B 12, with new neuronal circuits (dotted line).

The goal is to utilize the SgC 4 to cause an increase in neck stiffness as an automatic and reflexive activity. In the present invention, an effective SgC 4 should elicit a co-contraction of most of the major muscles in the head-and-neck, as diagrammed in FIG. 3, thereby causing significant increases in neck stiffness.

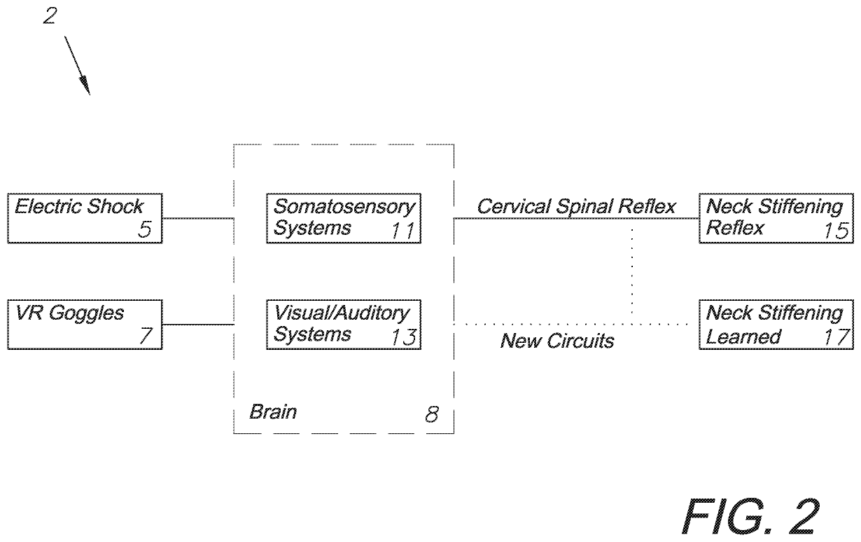

In one exemplary embodiment of the invention as shown in FIG. 2, the SgC 4 is delivered to the person being trained via a stimulation device 60, which may be a neck collar or other suitable device, capable of generating a mild electric shock 5 to the person being trained. Regions of the brain 8 which may be involved with learning the LB 16 associated with neck stiffening 17 based upon the neck stiffening reflex 15 include the somatosensory systems 11 and visual/auditory systems 13. The magnitude of the electric shock 5 is such that it is sufficiently unpleasant or threatening to elicit contraction of the extensors and flexors of most of the major head-and-neck muscles, but not sufficient to cause significant discomfort or apparent injury. The purpose of the SgC 4 is to cause neck stiffness via a reflex pathway already built-in as diagramed in FIGS. 1-2. A second purpose of the SgC 4 is to attach a significance for the SnC 6 to the person being trained when SnC 4 is presented to the person together.

In the present invention, one embodiment of the SgC 4 can take the form of a stimulus for stretch reflexes. Activation of stretch reflexes generally leads to an increase in muscle tone via reflexive muscular contractions. One example of the stretch reflex is the knee jerk reflex elicited by hitting the kneecap with a small hammer, as is sometimes done in the doctor's office. The strike of small hammer is typically on or near one of the points of insertion for the muscle quadriceps.

In the present invention, there may be, however, potential difficulties with the stretch reflex approach for SgC 4. Chief among these difficulties is the fact that the points of insertion for the muscles in the head-and-neck are deep and often not easily accessible and are often located diffusely over a large region of the body as is diagrammed in FIG. 3.

Besides the stretch reflex, the SgC 4 can take the form of a stimulus for the nociceptive reflex or withdrawal reflex. One example of the nociceptive reflex is the withdrawal of a limb elicited by a pinprick, as is sometimes done in the doctor's office. In the present invention, as stated earlier, a mild but effective electric shock 5 to the neck in order to elicit a reflexive, co-constriction of major extensors and flexors of the head-and-neck as R 14.

Besides the stretch reflex, the nociceptive reflex, and the vestibulocolic reflex approaches, the SgC 4 can take the form of a Virtual Reality (VR)-delivered stimulus for the activation of a neck-tensing reflex. For one example, this VR-delivered stimulus can be the unexpected appearance of a large hand pressing against the forehead of the glabellar area of the person delivered via VR goggles 7. In preliminary testing on human volunteers, we knew that such VR-delivered stimulus (without anything physically touching the human volunteer), the human subject can execute a neck-tensing behavior accompanied by a body-tensing behavior. In extreme cases the human subject actually can fall to the ground, believing that the VR-delivered glabellar push was so powerful that it pushed the volunteer to fall to the ground.

The effectiveness of the VR-delivered SgC 4 has its basis in the role of many posture-control or other reflexes. For example, built-in stretch reflexes are important for postural control. Stretch reflexes are present in most skeletal muscles and are evolved for the purpose of maintaining status quo—skeletal muscles prefer to stay put in their length and tension. Any attempt to change that will be met by a reflex to restore that status quo. Hence, a perceived glabellar tap or push will be met by the increase in neck stiffness to resist that push, albeit a VR-delivered “push.” Similarly, built-in vestibule-ocular reflexes (VOR) work to maintain the stability—status quo—of the visual field.

Besides the stretch reflex, the nociceptive reflex, or the VOR approaches, the SgC 4 can take the form of a stimulus for the activation of the vestibulocolic reflex which opposes head movements via head-and-neck muscular contractions.

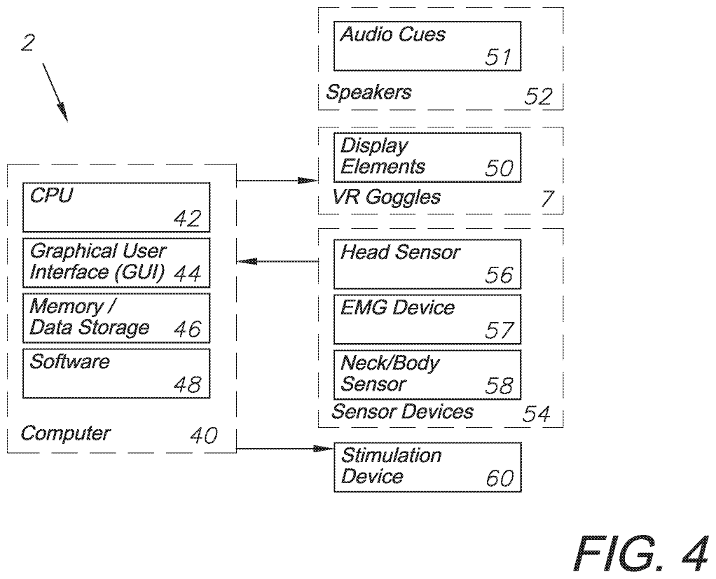

As shown in FIG. 4, an embodiment of this invention uses a computer 40 hooked up to VR Goggles 7, various sensor devices 54, an optional stimulation device 60 (which may be the VR Goggles 7), and speakers 52. The Computer, which includes a CPU 42, graphical user interface (GUI) 44, memory/data storage 46, and appropriate software 48, provides the stimuli which is sent to the VR Goggles 7 and speakers 52, controls any additional stimulation device 60, such as an electric shock device, and receives data back from the various sensor devices 54, after which LB is determined as true or false based on the results.

in one embodiment of the invention, visual images, displayed through the displayed element 50 within the VR goggles 7 or other suitable device, designed as SnC 6, will be modified so as to cause the person being trained to experience a dyscongruent sensation. For example, the images to the two eyes via the VR goggles 7 may be dyscongruent to each other which then activate the vestibulocolic reflex. In so doing, SnC 6 and SgC 4 are presented as one. The purpose is for this dyscongruent sensation to immediately elicit an increase in neck muscle tone via the neck stiffening reflex 15 from the activation of the vestibulocolic reflex.

In one embodiment of the invention, auditory cues 51 such as a loud yell from a coach may be employed as SgC 4 in order to generate the R 14 via an auditory startle reflex and played through speakers 52 which may be built into the VR goggles 7 or a separate set of speakers, such as in headphones or ear buds.

In one embodiment of the invention, many of the choices for SgC 4 described above may be employed together.

To summarize the description of the present invention up to this point, a number of SgC 4 may be used leading to a quick, subconscious, and reflexive increase of head-and-neck muscle tone.

The SnC 6 starts off as a neutral stimulus. In other words, the naïve person being trained would not reflexively stiffen his or her neck when the SnC 6 is presented for the first time. With time, and with repeated pairing of the SnC 6 with an effective SgC 4, the person gradually learns to attach a significance to the SnC 6 and will no longer treat the SnC 6 as a neutral stimulus. The immediate result is that the person being trained will generate an appropriate LB 16 quickly and subconsciously upon the presentation of the SnC 6. Moreover, the LB 16 so generated will be similar in function, timing, and morphology to R 14—also an increase in head-and-neck muscle tone. In contact sports, the desirable LB 16 will therefore generally be a quick and subconscious stiffening of his or her neck muscles 17 when a member of the opposing team is within striking range.

In the present invention, a major difference between R 14 and LB 16 is in timing. R 14 is generated by a hard-wired neuronal circuitry supporting a reflex. The timing, speed, or the latency of the reflexive muscular contractions are therefore, more or less, fixed with little or no room for modification. The LB 16, however, is a new and learned behavior which is generated by new neuronal circuits. The timing, speed, or the latency of the learned behavior is according to and prescribed by the manner in which the SgC 4 and SnC 6 are paired or coupled as shown in FIG. 1. In general, LB 16 occurs bearing a fixed relationship in timing to SnC 6, e.g. by a delay of ˜250 milliseconds.

In one embodiment of the invention, the SnC 6 as auditory 51 and visual 50 stimuli delivered to the person being trained via a device that comprises of special-purpose VR goggles 7. For example, one such effective SnC 6 could be a football player of the opposite team rushing toward you or a soccer player of the opposite team trying to head the ball at the same time as you are trying to head a ball.

In one embodiment of the invention, the VR device 7 of the present invention repetitively pairs and presents the SnC 6 (e.g. images of a football player rushing toward the person being trained via VR device 7) and the SgC 4 (e.g. a mild electric shock 5 to the neck) to the person being trained during the training sessions.

In one embodiment of the invention, the VR device 7 of the present invention does not simply repeated present identical visual images to the person being trained. Rather, the images will generally be similar but not identical. For example, the images may be taken from a compilation of literature database on how football players or soccer players or in other sports actually got injured [Comstock R D, Currie D W, Pierpoint L A (2015) SUMMARY REPORT. NATIONAL HIGH SCHOOL SPORTS-RELATED INJURY SURVEILLANCE STUDY. 2014-2015 School Year. www.ucdenver.edu/academics/colleges/ . . . /Original%20Report_%202014_15.pdf], or otherwise depending upon the specific sports of the person being trained.

For example, in one embodiment of the invention, the SnC 6 will comprise of visual images such as a short segment of video (e.g. lasting a fraction of a second). The short segment of video will be taken from a compilation of such videos, which collectively represent how football players or soccer players or athletes in other sports actually got injured [Comstock R D, Currie D W, Pierpoint L A (2015) SUMMARY REPORT. NATIONAL HIGH SCHOOL SPORTS-RELATED INJURY SURVEILLANCE STUDY. 2014-2015 School Year. www.ucdenver.edu/academics/colleges/ . . . /Original%20Report_%202014_15.pdf].

In one embodiment of the invention, for example, the collection of such visual images will be a statistical representation of how football players or soccer players actually got injured (e.g. 30% from body to body contacts, 30% from head to body contacts, 30% from head to ground contacts, and 10% from body to ground contacts.

In general, the athlete being trained has one sport which he or she is engaged in. In one embodiment of the invention, the collection of such visual images will be a statistical representation of how the athletes in that sport actually were injured. For example, for athletes engaged in football, soccer, hockey, basketball, etc., the sources of SnC can be designed accordingly.

The timing of the SnC 6 is such that it should be delivered, optimally, at about a faction of a second, (e.g. 250±100 milliseconds) before the impact [Christian K M and Thompson R F (2003) Neural substrates of eye blink conditioning: Acquisition and retention, Learning and Memory 10:427-455. doi: 10.1101/lm.59603]. This delay may be increased or decreased with consideration of the particular sport.

The delay is also designed with a concept of defining a personal safety zone. For example, in the game of football, it is possible to have two players from the opposite team running toward each other at full speed. If the full speed is approximately 10 meters per second, the two players are moving closer at a rate of 20 meters per second or 20 meters per 1000 milliseconds. In 250 milliseconds, a gap of 5 meters will disappear and an impact is likely to occur. Therefore, 250 milliseconds delay will teach the person being trained to mentally define a personal safety zone of approximately 5 meters in radius. This is consistent with the SnC 6 being designed at about 250 milliseconds before impact.

The timing of the SnC 6 is such that it should be delivered at about [250±100 milliseconds] before the SgC 4. This delay is also consistent with the physiological activation of the head-and-neck musculature as a learned behavior. With respect to the activation of head-and-neck muscles, the fastest muscular contraction can occur within 100 milliseconds of the stimulus which is activating the muscular contraction as a reflex. The design of a 250 milliseconds delay therefore affords plenty of time for the learned behavior 16 to be on-line.

In the present invention, the purpose of the training is for the brain to form new neuronal circuits and generate the desired LB 16 which is similar in appearance and morphology to R 14 but with an appropriate timing according to and prescribed by the SnC 6 in order to be an effective countermeasure to the potential consequences of head impact as is diagrammed in FIG. 1. Therefore, the delivery of an exemplary SnC 6 will be integrated with the delivery of an exemplary SgC 4. In particular, the delivery of SnC 6 and SgC 4 will be synchronized with the SnC 6 always ahead of the SgC 4 by a fraction of a second, for example, 250 milliseconds with an optimal range between 150 to 350 milliseconds.

To summarize the discussion up to this point, the VR system 7 and device, including the VR goggles, are therefore implemented with capabilities to deliver a plurality of SnCs 6.

The system and device are also designed with capabilities to measure and monitor a plurality of R 14 and LB 16. Measuring and monitoring of the R 14 and LB 16 with the device and systems integrated structurally and functionally with the VR goggles 7 streamline the interaction between technology and training. Measuring and monitoring R 14 and LB 16 with a computer 40, such as a mobile device, allows the person to move freely in order to imitate the athlete in the field.

In one embodiment of the invention, R 14 is therefore a reflexive contraction of the extensors and flexors of most of the major head-and-neck muscles, which cause elevated neck stiffness.

The desired LB 16 is similar to R 14 caused by the SgC 4. Both R 14 and LB 16 involve an increase of the neck stiffness via contractions of the head-and-neck muscles. The LB 16, however, is a learned response, not a reflexive activity.

In one embodiment of the invention, R 14 is therefore a reflexive contraction of the extensors and flexors (or agonist and antagonist muscles) among most of the major head-and-neck muscles, which cause elevated neck stiffness.

In one embodiment of the invention, both R 14 and LB 16 are being measured and monitored by a system or device that is part of the training hardware, such as the computer 40, throughout the period of the training. In the very beginning of the training period, the person to be trained is naïve and does not know to stiffen his or her neck immediately before a head impact event. After the training, the person being trained will automatically and subconsciously stiffen his or her neck immediately before a head impact event occurs.

In one embodiment of the invention, one such measurement and monitoring method may involve the real-time measurement and monitoring of neck muscle contraction by EMG or electromyogram. A mobile EMG device 57 may be built into the VR goggle 7. The EMG electrode will be placed on the back of the neck of the person with a lead plugged into the VR device 7 which contains the EMG unit.

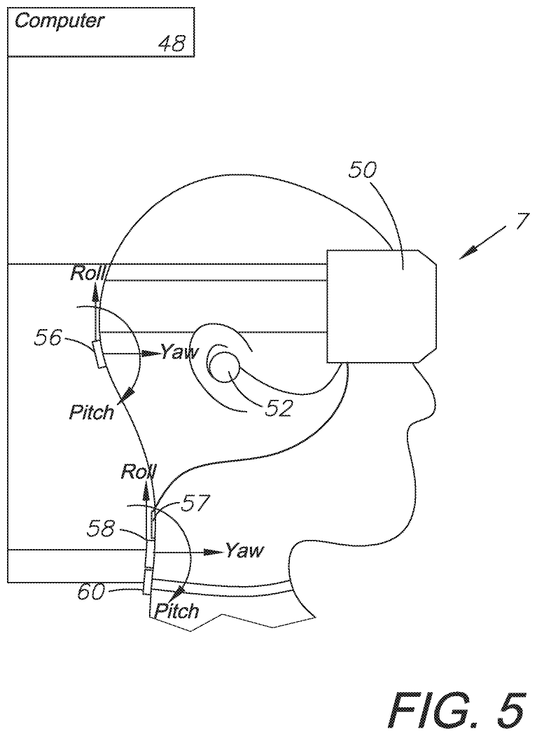

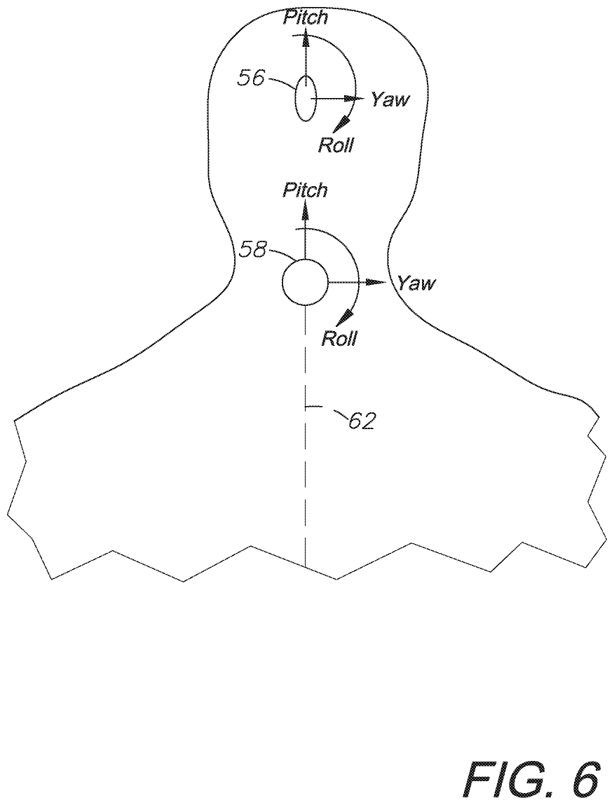

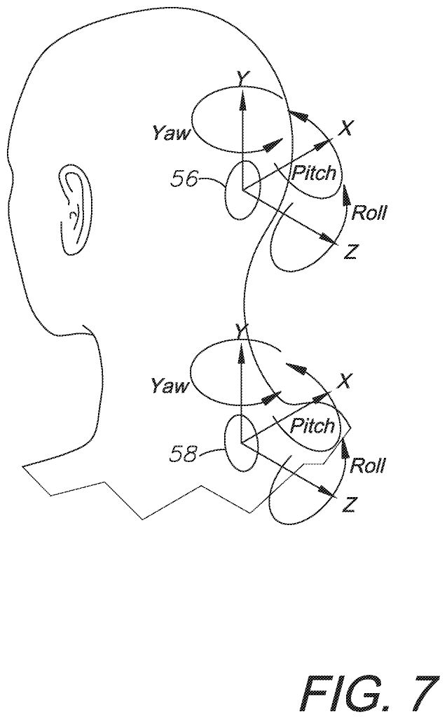

In one embodiment of the invention, one such measurement and monitoring method may involve the real-time measurement and monitoring of neck stiffness. To monitor neck stiffness in real time, we have developed a two-sensor system. A first sensor 56 is placed on the head to measure head movements and a second sensor 58 placed at the base or back of the neck along the spine 62 (between the C7 vertebra and the T1 vertebra) to measure body movements as shown in FIGS. 5-7. The first sensor 56 may be built into the VR goggle 7 or physically attached thereto, and the second sensor 58 may also be built into the VR goggle and connected as above. If the head-and-neck is stiff, the output of the head and the body sensor will be nearly identical. In human subjects, the head sensor generally registers larger velocities, accelerations, etc. We define dynamic neck stiffness index (DNSI) as the ratio of the output of the body sensor to that of the head sensor. In this way, our sensor monitors DNSI in real time, right up to the moment of a head impact event. DNSI is a vector with pitch, roll, and yaw components (the three degrees of rotational freedom of the human head-and-neck). It is also dynamic (sampled at 1 kHz) as DNSI must, from time to time, be reduced to near zero in voluntary head movements such as a soldier scanning the surroundings for enemy fire or a quarterback scanning the field for receivers.

Stiffness is a measure on how head-and-neck resists force. In the laboratory, it is tedious to measure neck stiffness, let alone monitor it in real time and at a high sampling rate, i.e. 1 kHz. The following data set is the result of a simulated study in our laboratory involving laboratory dummies and accelerometers.

A typical impact involves impact-induced head movements characterized by head angular accelerations (HAC). Data on HAC is expressed in our laboratory as pitch, roll, yaw components—the three rotational degrees of freedom in the human head-and-neck. Pitch is nodding the head to gesture ‘yes.’ Yaw is the head rotation to gesture ‘no.’ Roll is the third degree of freedom as in bending the head toward the left or the right shoulder.

To simplify, let us assume that the impact force in the example causes a pitch-type head rotation. The readout from the first sensor 56 and the second sensor 58 are listed in the following table as a timed series. In addition, the computer 48 calculates the ratio between the output of the second sensor 58 divided by the output of the first sensor 56 as DNSI. Let us also assume that the first sensor 56 samples the head angular acceleration (HAC) in pitch at a rate of 1 kHz. And let us also assume that the second sensor 58 samples the body angular acceleration (BAC) in pitch at a rate of 1 kHz. The data from the first sensor 56 and the second sensor 58 is also “sync'ed.”

A simulated readout is presented below.

| Time (msec) | HAC pitch | BAC pitch | DNSI |
|---|---|---|---|
| 0 | 0 | 0 | |
| 1 | 1 | 0.5 | 0.5 |
| 2 | 2 | 1 | 0.5 |
| 3 | 3 | 1.5 | 0.5 |
| 4 | 4 | 2 | 0.5 |
| 5 | 5 | 2.5 | 0.5 |
| 6 | 6 | 3 | 0.5 |
| 7 | 6 | 3 | 0.5 |
| 8 | 5 | 2.5 | 0.5 |
| 9 | 5 | 2.5 | 0.5 |
| 10 | 4 | 2 | 0.5 |
| 11 | 4 | 2 | 0.5 |
| 12 | 4 | 2 | 0.5 |
| 13 | 3 | 1.5 | 0.5 |
| 14 | 3 | 1.5 | 0.5 |
| 15 | 3 | 1.5 | 0.5 |
| 16 | 2 | 1 | 0.5 |
| 17 | 2 | 1 | 0.5 |
| 18 | 2 | 1 | 0.5 |

In the above example, we assume that the impact event starts at 0 msec. The values of HAC and BAC are in the same units such as degrees/sec2. The exact units of HAC and BAC are not relevant in this illustration. In this example, HAC and BAC first increases and then decreases. The impact-induced HAC and BAC are largely over within 20 msec of the impact, consistent with experimental data. [Rowson S, Brolinson G, Goforth M, Dietter D, Duma S (2009) Linear and angular head acceleration measurements in collegiate football, J Biomech Eng 131(6): 061016. doi: 10.1115/1.3130454] The DNSI stays at 0.5 throughout the impact event.

In the above example, the impact started and finished within ˜20 msec, a time period that is too short for any reflexes to come online. No human reflex can occur that fast (typical head-and-neck muscular reflexes have a latency nearly 100 msec). Therefore, human reflexes are too slow to play a role in mitigating concussion risk. The only exception is that if the athlete in question has a thick neck such as its static stiffness will result into smaller values of HAC compared with another athlete with a slender neck.

In the above example, we assumed that the impact event is a pitch head rotation. In practice, first sensor 56 and second sensor 58 samples information from pitch, roll, and yaw. The resultant DNSI generated is a vector, having pitch, roll, and yaw components.

With our CAT, we hope to accomplish the following. Our CAT will allow the athlete to learn to anticipate an imminent head impact event approximately 100-200 msec before the actual impact. The key is to anticipate the impact and activate the neck-tensing reflex so that the neck tensing occurs just prior to the impact in order to mitigate or reduce concussion risk.

Having described how the DNSI data is processed. We now can instruct the sensors to process DNSI and sent the data to the VR device 7 in a similar fashion as in the case of EMG data collection.

This system of angular acceleration detectors process angular accelerations of the head and the body. The system then analyzes and decomposes the head and body accelerations into angular accelerations in pitch, roll, and yaw. The system then computes quotients of the angular accelerations of the body and the head, pitch-to-pitch, roll-to-roll, and yaw-to-yaw. This computation generates three numbers which we refer to as the neck stiffness indexes (NSI) in pitch, roll, and yaw or (NSIP, NSIR, NSIY). The system also computes NSI as the vectorial sum of NSIP, NSIR, and NSIY.

In one embodiment of the invention, values of NSIP, NSIR, NSIY, and NSI are displayed as a function of time during the training sessions. It is to be expected that values of values of NSIP, NSIR, NSIY, and NSI will gradually increase during the course of training.

Once the person has learned to stiffen his or her neck upon an impending head impact event, the person is deemed trained for concussion avoidance. He or she may then practice and play the game or the sport without wearing any of the system or device used in the training.

To summarize the discussion up to this point, the VR system 7 and device, including the VR goggles, are therefore implemented with capabilities to measure and monitor a plurality of R 14 and LB 16, which are generally in the form of a dynamic increase in head-and-neck muscle tone as well as neck stiffness.

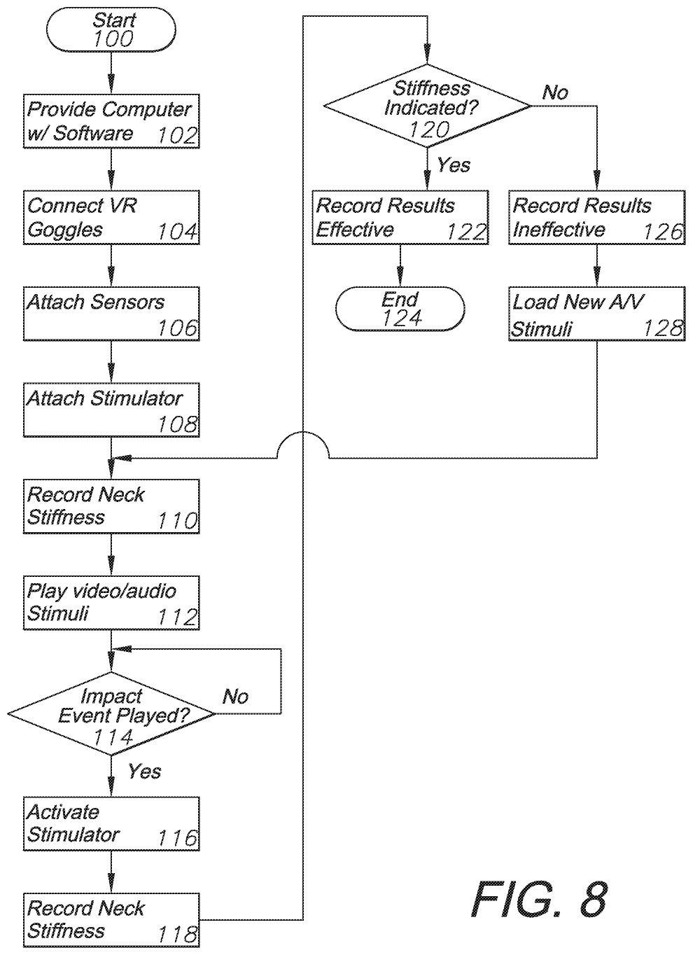

FIG. 8 steps through the process of collecting the data and teaching the LB 16 through the equipment as discussed above. The process starts at 100 and the computer with appropriate software is provided at 102. VR system 7, including VR goggles, are connected to the computer at 104, either through a wireless or wired connection. The various sensor devices 54 are attached to the user at 106. The stimulator 60, if used, is attached at 108.

The system records the user's neck stiffness at 110 to generate a baseline. The video/audio stimuli forming the SnC 6 and SgC 4 are played or initiated to the user at 112. The video and audio stimuli continue until the impact event is played at 114. Once this occurs, the video itself or in combination with the simulator 60 at 116 should result in a reflex contraction 14. The neck stiffness of the user is recorded at that point as well at 118.

The system determines if the user stiffened at 120 appropriately to cause LB 16. If yes, results are recorded as effective at 122 and the process ends for now at 124. Otherwise the results are recorded as ineffective at 126 and new audio/video stimuli may be loaded and played at 128, continuing the process until behavior is learned.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A concussion avoidance training (CAT) system for generating learned behavior, the system comprising:
a sensory cue;
a significance cue;
a computer comprising a processor, data storage, and graphical user interface;
an interactive interface configured to deliver said sensory cue and said significance cue;
a first sensor configured to measure three-dimensional roll, yaw, and tilt about said interactive interface;
a second sensor configured to measure muscle contractions; and
said computer configured to determine if a learned behavior results from application of said sensory cue and said significance cue through said interactive interface.

2. The system of claim 1, further comprising:
said interactive interface comprising a virtual reality (VR) headset having a visual display for presenting images and video as sensory cues or significance cues; and
said cues comprising a video presented on said visual display.

3. The system of claim 1, further comprising:
at least one speaker configured to provide audible audio cues; and
said significance cue comprising an audio cue produced by said at least one speaker.

4. The system of claim 1, further comprising:
an electric shock stimulus device configured to be placed in proximity with said interactive interface; and
said electric shock stimulus device configured to produce said significance cue in the form of an electric shock.

5. The system of claim 1, further comprising:
a stimulus device configured to generate VR-based glabellar push or tap configured to be placed in proximity with said interactive interface; and
said glabellar stimulus device configured to produce said significance cue in the form of a glabellar stimulus.

6. The system of claim 1, further comprising:
a stimulus device configured to generate VR-based VOR-related cues configured to be placed in proximity with said interactive interface; and
said VOR-related stimulus device configured to produce said significance cue in the form of a VOR stimulus.

7. The system of claim 1, further comprising:
a stimulus device configured to generate assorted relevant video sensory cues configured to be placed in proximity with said interactive interface; and
said assorted video sensory stimulus device configured to produce said sensory cues in the form of video signals.

8. A method of training learned behavior, the method comprising the steps:
providing a computer having a processor, data storage, and graphical user interface;
providing an interactive interface and controlling said interactive interface with said computer;
providing a first sensor and placing said sensor in proximity with said interactive interface;
producing a significance cue;
producing a sensory cue;
measuring the head and body movements in three-dimension and in real time with said interactive interface and first sensor;
measuring muscle contraction with a second sensor; and
determining, with said computer, whether a learned behavior indicated by elevated neck stiffness occurs.

9. The method of claim 8, further comprising the steps:
wherein said interactive interface comprising a virtual reality (VR) headset having a visual display for presenting images and video; and
presenting said significance cue and said sensory cue on said visual display.

10. The method of claim 8, further comprising the step:
producing a significance cue by generating audio from at least one speaker.

11. The method of claim 8, further comprising the steps placing an electric shock stimulus device in proximity with said interactive interface; and
producing said sensory cue from said electric shock stimulus device.

12. A method of training learned behavior, the method comprising the steps:
provide a computer comprising a processor, data storage, and graphical user interface;
connecting a virtual reality (VR) headset to said computer and deploying said VR headset;
attaching a first sensor configured to detect head movement in pitch, roll, and yaw;
attaching a second sensor configured to detect muscle contractions;
recording a first neck stiffness value with said computer based upon output of said second sensor;
providing stimuli via said VR headset;
analyzing data from said first sensor and said second sensor with said computer; and
recording neck stiffness, based upon recording motion in pitch, roll, and yaw as detected by said first sensor, with said computer based upon output of said first sensor and said second sensor; and
making a determination of learned behavior indicated by elevated neck stiffness with said computer.

13. The method of claim 12, further comprising the steps:
connecting a stimulus device for presenting a significance cue; and
providing said significance cue via said VR headset.

14. The method of claim 12, further comprising the steps:
connecting a stimulus device for presenting sensory cue; and
providing said cue via said VR headset.

15. The method of claim 12, further comprising the steps:
of providing a third sensor configured to detect body movements in pitch-roll-yaw; and
y calculating the neck stiffness index with said computer.

16. The method of claim 12, further comprising the steps:
of determining the extent of elevation of neck stiffness during the progression of the concussion avoidance training (CAT) with said computer.
